(19) United States
(12) Patent Application Publication
(10) Pub. No.: US 2003/0027150 A1
(43) Pub. Date: Feb. 6, 2003

(54) Method of haplotyping and kit therefor

(76) Inventor: David A. Katz, Evanston, IL (US)

Correspondence Address: STEVEN F. WEINSTOCK; ABBOTT LABORATORIES, 100 ABBOTT PARK ROAD, DEPT. 377/AP6A, ABBOTT PARK, IL, 60064-6008, US

(21) Appl. No.: 09/922,277

(22) Filed: Aug. 3, 2001

(51) Int. Cl. C12Q 1/68; C12P 19/34

(52) U.S. Cl. 435/6; 435/91.2

(57) ABSTRACT

A method of identifying the haplotype of an organism comprising the use of multiple duplexed amplifications of a single copy of an isogenic nucleotide sequence of interest coupled with detection of putative nucleotide sequence polymorphisms, as well as a kit and apparatus therefor.

### FIELD OF THE INVENTION

The present invention relates to the field of genetics. More specifically, the present invention relates to a method of haplotyping an organism. The invention has utility in medical therapeutics (including, but not limited to, establishment of drug dosing parameters), forensics, disease screening, as a tool for studying haplotypic/phenotypic relationships, and other areas.

### BACKGROUND OF THE INVENTION

For any particular DNA sequence or gene, a “normal” or consensus sequence for a population can be identified, and any particular individual in that population can have DNA containing nucleotide sequence insertions, deletions, and/or changes, which are commonly called “variants.” When a number of variants are located at substantially the same location in an organism's genome, this collection of two or more variants is known as a polymorphism. The chromosomes of organisms that reproduce sexually are paired (a partial exception is the X-Y chromosome “pair” in mammalian males). Accordingly, such organisms' genomes generally have two copies of every DNA sequence or gene.

These two copies, or “alleles,” may or may not be identical in a single organism. When two or more nucleotide sequence variants occur within a particular DNA sequence or gene, each allele is known as a “haplotype.” It is often useful to identify the haplotypes in an individual, for example, to appropriately diagnose a condition of the individual.

For example, a number of polymorphisms in the human thiopurine methyltransferase (TPMT) gene are known. These polymorphisms lead to a number of haplotypes. Four of these TPMT haplotypes are TPMT * 1, TPMT *3A, TPMT *3B, and TPMT *3C. The haplotype combinations * 1/*3A and *3B/*3C cannot be distinguished from each other by standard genetic testing procedures, but the ability to determine which TPMT haplotype combination exists in an individual is important because certain drugs such as azathiaprine are clinically tolerated in *1/*3A individuals, but cause serious adverse effects, including possible death, in *3B/*3C individuals.

Currently available technologies for distinguishing between these (and other relevant) haplotypes are inadequate or significantly inconvenient and slow. In this regard, Dr. Richard Weinshilboum of the Mayo Clinic (a leader in the field of TPMT genetics) has referred to current technology for haplotyping clinical patients as impractical, and has repeatedly called for improved methods to aid clinicians (including at at least the last two annual American Society for Clinical Pharmacology and Therapeutics meetings). Two methods of identifying genetic information relevant to a particular organism are disclosed by Vogelstein et al. and by Michalatos-Beloin et al.

Vogelstein et al., Proc. Nat'l Acad. Sci. (USA), 96, 9236-9241 (1999) discloses a method of identifying somatic mutations of the DNA of single cells in a population of cells. According to Vogelstein et al., DNA can be extracted from a population of cells that are suspected of comprising cancerous cells. The DNA is diluted into a multi-well test plates such that, on average, each well comprises less than about one genome equivalent of an gene sequence of interest. Using simple statistical methods, the number of test wells expected to contain a single copy of the gene of interest can be identified. This information is then used to predetermine a number of test wells to be tested. PCR is then used to amplify these single copies of the cellular DNA in the predetermined number of test wells. The amplified DNA in each test well has a uniform sequence because all the DNA was amplified from a single nucleic acid. These amplified DNA sequences are individually probed for mutations associated with the suspected cancer. Accordingly, nucleotide sequences indicating cancer can be identified even though these nucleotide sequences are present in only a very small portion of the cells tested. Vogelstein et al. refer to this technology as “Digital PCR.” Vogelstein et al. neither suggests that this technology is applicable to haplotyping, nor does Vogelstein et al. explain how to adapt this technology to haplotyping. Thus, the Vogelstein et al. method does not solve the long-felt need identified by Dr. Weinshilboum, particularly as applied to TPMT genetics.

Michalatos-Beloin et al., Nucleic Acids Research, 24, 4841-4843 (1996) discloses a method of molecular haplotyping that employs the use of allele-specific long-range PCR. This method employs PCR to generate products that are multiple kilobases long and requires the use of PCR primers that are specific for individual alleles (allele-specific PCR). The use and development of allele specific primers is expensive and can cause difficulties including, but not limited to, low reproducibility. Moreover, the method disclosed by Michalatos-Beloin is useful only to detect polymorphisms that are relatively close to each other so that a single PCR reaction can amplify more than one putative site of a polymorphism. The two relevant polymorphisms in TPMT are more than 10 kb apart. Accordingly, the method disclosed by Michalatos-Beloin et al. is not well suited to solve the long-felt need identified by Dr. Weinshilboum, particularly as applied to TPMT genetics.

Other techniques for distinguishing haplotypes include the combination of subcloning and DNA sequencing and inferential family studies.

The combination of DNA subcloning and sequencing is slow and expensive and can have other limitations. Accordingly, while DNA subcloning and sequencing might conceivably be suitable for the determination of some haplotypes, it is inconvenient and not a generally useful tool for the determination of haplotypes, nor for distinguishing the *1/*3A TPMT haplotype from the *3B/*3C TPMT haplotype in humans.

If the genotype of a sufficiently large number of members of a family can be determined, it is frequently then possible to determine haplotypes for members of the group by inferential methods. Clearly, such methods are not well-suited to meeting the long-felt need identified by Dr. Weinshilboum, particularly as it pertains to identifying the haplotype combination for individuals in most clinical situations, as genotyping family members is impractical (sometimes impossible) and often raises ethical concerns.

Thus, a long-felt need exists for a rapid means to establish the haplotype of an organism, both at the TPMT locus and at other loci in an organism's genome. A solution for this need would preferably be amenable to automation and be consistent with the needs of clinical diagnosis and/or treatment.

### BRIEF SUMMARY OF THE INVENTION

The present invention provides a method of identifying the haplotype of an organism with respect to a locus having isogenic sequences that comprises at least two possible polymorphisms at the locus. The method comprises aliquotting into discrete test locations nucleic acids obtained from the organism, or having counterpart sequences to those of the organism, that contain isogenic nucleotide sequences of interest. The aliquotting is performed such that there is a substantial probability that a number of test locations will contain exactly one isogenic nucleotide sequence with respect to the polymorphic locus of interest. The nucleic acids in the discrete test locations are then amplified to facilitate detection of specific alleles in each (amplified) nucleic acid. The presence or absence of specific alleles is detected in the isogenic region of interest in the amplified nucleic acids in each of a number of discrete locations in which amplification was performed. The specific alleles can be detected with a probe or other means. When this is performed in a sufficient number of test locations and under suitable conditions, the haplotype with respect to the locus of interest can be identified. Specifically, if a first specific allele is present in a test location only when a second specific allele is also present at a test location, then the two alleles must be present on the same chromosome. In contrast, if the first specific allele and the second specific allele are not typically located in the same test locations, then these alleles must be present on separate chromosomes. The detection of specific alleles can take place in the container in which the nucleic acids are amplified, or in other embodiments, the amplified nucleic acids can be transferred to other locations before the detection of the specific alleles is carried out. The locus of interest preferably has clinical implications that impact the diagnosis, or treatment, or both of the organism.

The present invention also provides a method for determining a human haplotype at the thiopurine methyltransferase locus consistent with the method described above.

The present invention also provides a kit useful for identifying the haplotype of an organism.

### DETAILED DESCRIPTION OF THE INVENTION

The present invention provides a method of identifying the haplotype of an organism that may have two or more genetic variants in a nucleotide sequence or at a genetic locus. The organism to be haplotyped preferably is known to have at least two genetic polymorphisms at a particular isogenic locus.

The inventive method of identifying a haplotype comprises providing a sample containing nucleic acids, and aliquotting the sample so that individual nucleotide sequences that may have variant nucleotide sequences can be individually amplified. A number of aliquots, which number is preferably predetermined, are then amplified. The amplification aids in the detection of specific alleles at each of the polymorphisms in each aliquot. The presence or absence of specific alleles in the (individually) amplified nucleic acid sequences in each of the assayed aliquots then allows the rapid and unambiguous determination of the organism's haplotype. Advantageously, the present inventive method is amenable to automation and can be performed with low cost compared to other known methods for determining a haplotype. Additionally, the method can incorporate the use of a computer product that performs automated haplotypic analysis and report generation.

The present inventive method comprises providing a sample from the organism. The sample contains nucleic acids obtained directly or indirectly from the organism. Nucleic acids suitable for use in the present invention preserve or reflect the distribution of nucleotide variants among the chromosomes (or other nucleic acid) carrying the genetic locus of interest irrespective of whether the nucleic acids are obtained directly or indirectly from the organism. Of course, the nucleotide sequences of interest are encoded by at least two isogenic regions of a pair of chromosomes in the organism's genome or other nucleic acids. Accordingly, the present inventive method typically does not include analysis of those portions of a mammalian Y chromosome that are not homologous to a region on another chromosome, e.g., the X chromosome.

While in the context of medical diagnosis, the putative polymorphisms are preferably located in a portion of a gene affecting gene function, the polymorphisms can also be located in intergenic regions of the chromosome as well. The use of intergenic polymorphisms are useful in many embodiments of the present inventive method, but are particularly useful for genotype-phenotype relationship discovery research, and in the context of forensic applications and investigations into genetically-based parent-child or similar familial relationships.

The sample can be obtained from any suitable source, such as for example, blood, eye fluid, cerebral spinal fluid, milk, ascites fluid, synovial fluid, peritoneal fluid, amniotic fluid, tissue, cell cultures, products of an amplification reaction and the like, environmental sources, and forensic sources including sewage and biological material deposited in or on cloth.

In some embodiments, the sample can be amplified directly as obtained from the source. Alternatively, the sample can be amplified following pre-treatment. For example, prior to amplification the test sample can be pre-treated to obtain, plasma from blood, substantially isolated cells from biological fluids, and/or a (tissue, cell, or other) homogenate. Similarly, the sample can be processed to prepare a liquid from a solid material, processed to inactivate interfering components, and/or concentrated (although the sample will more typically be diluted during the aliquotting step). The sample can also be processed to purify or partially purify the nucleic acids in the sample, and any suitable purification method can be employed to obtain purified or partially purified nucleic acids.

The sample provided in the first step of the present inventive method preferably comprises genomic DNA from the organism. Genomic DNA is preferable because it can be obtained directly from a wide variety of tissue sources, and can be subjected to amplification with a minimum amount of processing. Moreover, pre-treatment steps that are desirable or occasionally required to amplify genomic DNA from biological sources are well known in the art.

The sample can contain intact nucleic acids (i.e., as they exist in the organism's cells), or can contain fragments of the nucleic acids. In this regard, fragmented nucleic acids are preferably relatively large so that it is less likely that a break or shear will occur between nucleotide variants of interest, which can destroy the haplotypic information encoded or contained in a particular nucleic acid. Therefore, the nucleic acids of the sample preferably are not so degraded that the distance between the first and second nucleotide variants is greater than the median length of nucleic acid fragments in the sample. In this regard, if more than two putative nucleotide variants are to be detected, then the nucleic acids of the sample preferably are not so degraded that the median length of the nucleic acid is greater than the distance between the two nucleotide variants that are farthest apart from each other. Similarly, the sample is preferably processed, if at all, so as to avoid excessive and unsuitable shearing or breakage of the nucleic acids in the sample. In contrast, however, some nucleic acid shearing can be advantageous because of its effect on the fluid dynamics of the sample containing the nucleic acid. In any event, it is difficult to prevent entirely the shearing of large nucleic acids, and it is not necessary to entirely prevent such shearing. Suitable methods for obtaining nucleic acids directly or indirectly from organisms that produce nucleic acid fragments of suitable sizes are well known in the art.

Other sources of nucleic acids from the organism also can be used. For example, when two or more polymorphisms of interest are present in mRNA of the organism, the mRNA can be subjected to amplification. Of course, in some instances amplification of mRNA is more complicated than amplification of genomic DNA, and therefore, can be less preferred than the amplification of genomic DNA. On the other hand, the use of mRNA or CDNA or both can be preferred for multiple reasons including that use of mRNA and/or cDNA allows the skilled artisan, in the context of the present invention, to determine if RNA is transcribed preferentially from one or both alleles. The provided sample also can comprise cDNA, the preparation of which is frequently an initial step in the amplification of mRNA.

Advantageously, cloning of the nucleic acids derived from the organism is not required. Nonetheless, the nucleic acids optionally can be cloned prior to amplification or analysis. In that event, any suitable cloning vector can be employed. Suitable cloning vectors in the context of the present invention include viral-derived vectors (e.g., vaccinia viral vectors or adenoviral vectors), phage-derived vectors, bacterial artificial chromosomes, yeast artificial chromosomes, and other vectors. In some embodiments, the selection of the vector will depend in part on the known or suspected distance between the polymorphisms of interest, such that nucleic acid sequences of sufficient size can be cloned. Of course, the use of cloning can increase the cost and complexity of the inventive method.

Any suitable sample comprising nucleic acids in which the physical segregation of polymorphisms of interest among the chromosomes is maintained or reflected can be used in the context of the present inventive method.

A lysing reagent optionally can be added to the sample, particularly when the nucleic acids in the sample are sequestered or enveloped, for example, by cellular or nuclear membranes. Additionally, any combination of additives, such as buffering reagents, suitable proteases, protease inhibitors, nucleases, nuclease inhibitors, and detergents, can be added to the sample to improve the amplification and/or detection of the nucleic acids in the sample. Additionally, when the nucleic acids in the sample are purified or partially purified, the use of precipitation can be used, or solid support binding reagents can be added to or contacted to the sample, or other methods and/or reagents can be used. The ordinarily skilled artisan can routinely select and use additives for, and methods of, partial purification of the nucleic acids in the sample without little or no experimentation.

The sample, irrespective of whether it has been pre-processed is then aliquotted, i.e., small portions of the sample are placed into discrete test locations. Aliquotting the sample serves to distribute individual molecules comprising an isogenic region of interest into discrete test locations such that at least one test location contains a single copy or equivalent of the isogenic nucleotide sequence of interest. The portion of the original sample that is aliquotted into each physically discrete test location can be determined empirically or can be readily calculated by the skilled artisan. When the nucleic acid is aliquotted, the skilled artisan can calculate the portion of the sample to be distributed to each test location by considering, among other factors, the total genome size (e.g., in micrograms and base pairs), the quantity of DNA in the sample (e.g., in micrograms), and optionally, the fragment size distribution (e.g., in base pairs) of the DNA just prior to aliquotting. This process of aliquotting optionally can be referred to as “single molecule dilution.” Amplification of a single molecule having an isogenic sequence results in a detectable population of nucleic acids with a substantially uniform nucleotide sequence.

The sample is diluted or serially diluted in most embodiments of the present invention, which facilitates the process of aliquotting a sample comprising a single copy of the isogenic sequence of interest. While dilution is a useful step, it is not required, especially when the sample provided in the first step of the method is already relatively dilute (e.g., as in a forensic sample having a nucleic acid concentration of from about 0.2 to about 100 genome equivalents per aliquot volume, wherein aliquot volume means the volume of the sample in an amplification reaction according to the present invention; typically from about 25 nanoliters to about 3,000 microliters).

The amount of nucleic acid contained in each aliquot varies because ordinary transfer of very dilute solutions is inherently stochastic. Thus, it is appropriate to calculate and/or determine the average nucleic acid content in each aliquot. Each aliquot in the present inventive method preferably contains, on average, less than about 1.7 copies of the isogenic sequence of interest, because this simplifies the statistical treatment of data obtained from the present inventive method. More preferably, the amount of nucleic acid contained in an aliquot on average contains less than about 0.8 copies, and even more preferably contains less than about 0.6 copies. Similarly, each aliquot preferably contains, on average, at least about 0.1 copies of the isogenic sequence of interest, and more preferably, at least about 0.25 copies, and even more preferably, at least about 0.4 copies of the isogenic sequence of interest. The ordinarily skilled artisan can empirically determine the average amount of nucleic acid in each aliquot, inter alia, by observing the rate of aliquots containing no copies of the isogenic nucleotide sequences of interest in serial dilutions, no nucleic acids in serial dilutions, or by other methods known in the art.

When the amount of nucleic acid in each aliquot approaches, on average, 0.5 copies (of the isogenic nucleotide sequence of interest) per aliquot and a Poisson-like distribution is obtained, most test locations will contain 0 or 1 copy of the isogenic nucleotide sequence. Accordingly, for any two nucleotide sequence variants, three possibilities arise for the organism to be haplotyped. In the first possibility, a specific allele of a first polymorphism and a specific allele of a second polymorphism are always (or statistically and substantially always) detected in the same test locations. This result indicates that the specific alleles occur on the same chromosome or nucleic acid. In the second possibility, a particular specific allele of the first polymorphism is observed only in test locations where a specific allele of the second polymorphism is absent. This result would indicate that the nucleotide sequence variants are on separate chromosomes or nucleic acids. In the third possibility, the specific allele of the first polymorphism is observed at about one-half the rate of the specific allele at the second polymorphism and substantially only in the test locations containing the second specific allele, whereas the second allele is observed in substantially the same number of wells as are predicted to contain detectable copies of the isogenic nucleotide sequence of interest. This third possibility indicates that the individual is hemizygous for the first specific allele of the first polymorphism and homozygous for the second specific allele of the second polymorphism.

The aliquotting procedure used in the present inventive method is similar to that discussed in Vogelstein et al., Proc. Natl. Acad. Sci. (USA), 96, 9236-9241 (1999), which uses an embodiment of the aliquotting technique of the present inventive method for non-haplotyping purposes. Additionally, while the present inventive method is explained in the context of a diploid gene, the skilled artisan readily can adapt this methodology to gene families.

The test locations into which each sample is aliquotted can be of any suitable form. Optionally, the test locations can be an array wherein separation of the test locations is maintained primarily by chemico-physical forces or electrical fields, e.g., surface tension or a hydrophobic lattice layered onto a hydrophilic surface. For ease of use, however, the test locations are optionally wells of a microtiter or microassay plate. The microtiter plate wells can be sealable or reversibly-sealable so as to provide a barrier against aerosol-transfer of nucleic acids and other forms of contamination. Moreover, the microtiter plate can be placed in a low pressure container or flow cell so that aerosols that form during the method can be removed from the vicinity of the test locations. In a yet more preferred embodiment, a multiplicity of test locations can be sealed using a thin adhesive film or other suitable film-like structure to provide isolated test locations. Optionally, samples and reagents can then be added to the test locations with a sharp pipette tip or canula, which optionally can be disposed after use, or decontaminated. There is no requirement that amplification and detection of nucleic acids occur in a single container or location.

Portions of the isogenic nucleotide sequence of interest in the aliquotted sample or processed aliquotted sample are then amplified via a duplexed or multiplexed amplification process in each of a multiplicity of test locations. Any suitable duplexed or multiplexed amplification process can be employed. Suitable amplification techniques include, but are not limited to, ligase chain reaction (LCR), e.g., as described in European Patent Number 320 308 and its variations (such as “gap LCR” described in U.S. Pat. No. 5,792,607 and “multiplex LCR” described in International Patent Application WO 93/20227), NASBA and similar reactions such as transcription-mediated amplification (TMA), e.g., as described in U.S. Pat. No. 5,399,491, Invader™ assays using for example a “cleavase” enzyme, and preferably polymerase chain reaction (PCR), e.g., as described in U.S. Pat. Nos. 4,683,195, 4,683,202, and 5,582,989. Suitable amplification techniques can also include, without limitation, Self-Sustained Sequence Replication (3SR) as described in Fahy et al., PCR Methods and Applications, 1, 25-33 (1991) and variations thereof, and strand-displacement amplification (SDA) as described in Walker et al., Proc. Natl. Acad. Sci (USA), 89, 392-96 (1992) and variations thereof such as Rolling Circle Amplification (RCA).

In general, the amplification process selected comprises adding amplification reaction reagents to a sample aliquot to form an amplification reaction. Nucleic acid sequences of interest in the sample aliquot are then amplified by maintaining the amplification reaction at a suitable temperature(s) for a suitable period(s) of time. Amplification reaction reagents suitable for use in nucleic acid amplification reactions are well known. Amplification reaction reagents can include, but are not limited to: a single or multiple reagent, one or more enzymes having reverse transcriptase, polymerase, and/or ligase activity; enzyme cofactors such as magnesium or manganese; salts; nicotinamide adenine dinucleotide (NAD); and deoxynucleoside triphosphates (dNTPs) such as, for example, deoxyadenosine triphosphate, deoxyguanosine triphosphate, deoxycytodine triphosphate and thymidine triphosphate. The skilled artisan can readily select appropriate amplification reaction reagents based upon the particular type of amplification reaction selected.

In the context of the present invention, a duplexed assay employs two pairs of oligonucleotides. The oligonucleotides of each pair of oligonucleotides hybridize either to the same strand of the isogenic polynucleotide of interest, when e.g., LCR is employed, or to opposite strands of the isogenic polynucleotide sequence of interest, when e.g., PCR, NASBA, or TMA is employed. Additionally, the oligonucleotides of a pair of oligonucleotides preferably do not overlap. The oligonucleotides can be of any suitable length and composition. However, the oligonucleotides are preferably selected to facilitate robust amplification of two (in the case of duplexed amplification) or more (in multiplexed amplification) regions of the isogenic nucleotide sequence of interest. Of course, the regions of interest are those regions of the nucleotide sequence that actually or potentially contain a sequence variant. Moreover, when the sample contains only a single stranded nucleic acid and a two-stranded (e.g., PCR), rather than a single-stranded (e.g., LCR), amplification technology is employed, then the second oligonucleotide is complementary to the nucleic acid produced by replicating the single-stranded nucleic acid.

Similarly, multiplexed amplification reactions can be used in the context of the present invention. Multiplexed amplification reactions employ three or more pairs of oligonucleotides so as to amplify three or more sites of putative nucleotide polymorphisms.

The amplification reaction can be employed for any suitable number of “cycles” in embodiments employing amplification processes that have sub-processes known as “cycles,” e.g., PCR. From about 10 to about 90 cycles are preferably employed, and from 45 to 75 cycles are more preferably employed in embodiments employing cyclical amplification processes.

Additionally, a booster step, the use of which is known in the art (see, e.g., Ruano et al., Nucleic Acids Research, 17, 5407 (1989)) can be employed to improve the reliability or accuracy or other desirable characteristics of the amplification reaction. Briefly, booster amplification steps employ an initial quantity of amplification reaction reagents, especially oligonucleotides, that is lower than the final quantity of amplification reaction reagents used in the amplification process. The initial lower quantity of reaction reagents decreases the likelihood of spurious amplification reactions that can occur when particularly low (e.g., about 0.5 target copies per amplification reaction - on average) quantities of target are present in an amplification reaction, or when a high quantity of nucleic acid sequences other than those of interest are present in the amplification reaction.

Similarly, in embodiments employing the polymerase chain reaction, any suitable set of amplification parameters can be employed. For example, the precise temperatures at which double stranded nucleic acid sequences dissociate, primers hybridize or dissociate, and polymerase is active, are dependent upon, inter alia, the length and composition of the sequences involved, the salt content of the reaction, the difference if any between the oligonucleotide sequence and the target nucleic acid sequence, the oligonucleotide concentration, the viscosity of the reaction, and the type of polymerase. The ordinarily skilled artisan can easily determine appropriate temperatures for the amplification reaction, usually with no or little experimentation (see, e.g., Wetmur, J. G., Critical Reviews in Biochemistry and Molecular Biology, 26, 227-59 (1991)). In this regard, temperatures above about 90° C., and preferably temperatures between about 92° C. and about 100° C., commonly are suitable for the dissociation of double stranded nucleic acid sequences. Temperatures for forming primer hybrids are preferably between about 45° C. and about 65° C., and more preferably between 55° C. and 59° C. Any suitable temperature can be selected for the polymerization or extension phase, however, the temperature is polymerization temperature is preferably between about 60° C. and about 90° C., and more preferably between about 70° C. and 80° C., because many thermostable polymerases are suitably active in this temperature range.

The distance between each actual, potential, or putative nucleotide sequence variants of interest is limited only by the shearing of the nucleic acid, particularly when aliquotting single molecules of the nucleic acid. The present inventive method is more advantageous than other potential prior art and non-prior art methods of determining haplotypes, however, when the distance between the nucleotide sequence variants is too great to be easily amplified in ordinary or ordinary-asymmetric amplification reactions that utilize two or three oligonucleotide primers, respectively. Accordingly, in embodiments of the present inventive method employing duplexed amplification, the distance between actual, potential, or putative nucleotide sequence variants can be greater than about 1,000 bases or bp, about 2,000 bases or bp, about 5,000 bases or bp, or about 10,000 bases or bp. Additionally, two actual, potential, or putative nucleotide sequence variants can be separated by structures or features that make non-duplexed/non-multiplexed amplification more challenging, less robust, or impossible. Such structures or features include, but are not limited to, strong stem-loop structures (for example in single stranded nucleotides), sites of high G-C content, sites with triplex-DNA formation potential, and strong-binding sites for nucleotide sequence binding proteins.

Optionally, the oligonucleotides hybridize to sequences flanking the putative polymorphic sites of the organism's genome such that less than about 1,200 bases or base pairs (bp), and more preferably less than about 600 bases or bp in length is amplified in a reaction using any particular pair of oligonucleotides. Preferably, two of the putative polymorphic sites amplified are separated in the organism's genome by more than 1,000 base pairs, preferably 2,000 base pairs, more preferably 3,500 base pairs, and yet more preferably, by more than 5,000 base pairs. The amplification products optionally can be sequenced, sub-cloned, or otherwise processed, which can be independent of their use in the identification of the organism's haplotype.

The skilled artisan can readily predetermine the number of test locations in which the isogenic nucleotide sequence of interest will be amplified. The number of test locations tested can be calculated in view of, among other possible factors, (i) the average length of the polynucleotides in the sample, (ii) the distance between the polymorphisms to be detected, and (iii) the percentage of test locations predicted to contain precisely one genome equivalent of the isogenic nucleotide of interest. The number of test locations to be tested can optionally also be predetermined in view of the symmetry or asymmetry of the polynucleotide length distribution. Additionally, the number of test locations in which the isogenic nucleotide of interest is amplified can be determined empirically, theoretically, or by a combination of empirical observation and theory. Nucleic acids in at least one test location that is expected to contain, or observed to contain, exactly one copy of the isogenic nucleotide sequence of interest is amplified. Preferably, nucleic acids in at least about three test locations expected to contain, or observed to contain, exactly one copy of the isogenic nucleotide sequence of interest are amplified. More preferably, nucleic acids in at least about six test locations expected to contain, or observed to contain, one and only one copy of the isogenic nucleotide sequence of interest are preferably amplified.

When the test locations contain an average of about 0.5 genome equivalents each, and the distribution of polynucleotides among the aliquots approaches a simple Poisson distribution, then one suitable number of test locations subjected to amplification is about ten test locations for many applications of the inventive method. For example, 10 wells containing an average of about 0.5 genome equivalents each would be expected to comprise 3 test locations that contain exactly one copy of the isogenic sequence of interest. 20 wells is another suitable number of test locations.

The amplified sample in each test location is then analyzed by any suitable method to determine which specific alleles of two or more polymorphisms are located in the amplified isogenic nucleotide sequence of interest. In this way the organism's haplotype is readily identified (i.e., the skilled artisan can readily identify whether two or more specific alleles are located on the same strand) according to the method discussed above. Of course, the amplified nucleic acid in some or each of the test locations optionally can be transferred to a new location or container prior to detection. A multiplicity of suitable methods to detect the specific alleles at polymorphic sites are known in the art, and the skilled artisan can readily select the method of detection most suited to a particular embodiment of the inventive method.

Suitable means include, but are not limited to, DNA sequencing (including e.g., Pyrosequencing™), Northern blotting, Southern blotting, Southwestern blotting, probe shift assays (see, e.g., Kumar et al., AIDS Res. Hum. Retroviruses, 5, 345-54 (1989), T4 Endonuclease VII-mediated mismatch-cleavage detection (see, e.g., Youil et al., Proc. Natl. Acad. Sci (USA), 92, 87-91 (1995)), Fluorescence Polarization Extension (FPE), Single Strand Length Polymorphism (SSLP), PCR-Restriction Fragment Length Polymorphism (PCR-RFLP), Immobilized Mismatch Binding Protein Mediated (MutS-mediated) Mismatch detection (see, e.g., Wagner et al., Nucleic Acids Research, 23, 3944-48 (1995), reverse dot blotting, (see, e.g., European Patent Application 0 511 559), hybridization-mediated enzyme recognition (see, e.g., Kwiatkowski et al., Mol. Diagn., 4(4), 353-64 (1999), describing the Invader™ embodiment of this technology by Third-Wave Technologies, Inc.), detection, single-strand conformation polymorphism (SSCP) and gradient denaturing gel electrophoresis to detect probe-target mismatches (e.g., “DGGE”, see, e.g., Abrams et al., Genomics, 7, 463-75 (1990), Ganguly et al., Proc. Natl. Acad. Sci (USA), 90, 10325-29 (1993), and Myers et al., Methods Enzymology, 155, 501-27 (1987)).

Preferably, however, the putative polymorphisms are detected by the use of an oligonucleotide probe that can be contacted to the amplification reaction in each test location to generate a signal that indicates the presence or absence of an allele of the polymorphic nucleotide sequence. Preferred means of detecting the nucleotide polymorphisms present in each test location include, but are not limited to, the use of paired detector-quencher probes wherein a detectable signal is amplified in the presence of a specific target nucleotide sequence (see, e.g., U.S. Pat. No. 5,928,862 to Morrison), the so-called TaqMan™ system (see, e.g., U.S. Pat. No. 5,210,015), and the use of so-called molecular beacons (see, e.g., U.S. Pat. No. 5,925,517), as well as variants thereof, and including both “real-time” and traditional formats. The molecular beacons can be employed in any suitable format, including formats that do require and do not require solid supports.

Oligonucleotide probes can form part of the initial reaction mixture or can be added in a separate step.

Thus, the probes can be used to detect the presence or absence of each specific allelic sequence in the amplification products (each in a discrete test location).

The probes optionally can be labeled with a first binding member that is specific for a binding partner that is attached to a solid support material. Similarly, oligonucleotide primers can be labeled with a second binding member specific for a conjugate, such as a binding member stably linked to a radioisotopes, fluorophores, chemiluminophores, nanobarcodes, enzymes, colloidal particles, fluorescent microparticles, fluorescence resonance energy transfer (FRET) pairs, and the like. The amplified nucleic acids of interest bound with the probes can then be separated from the remaining reaction mixture by contacting the mixture with the solid support and removing the solid support from the reaction mixture. Any probe/amplification product hybrids bound to the solid support can then be contacted with a conjugate to detect the presence of the hybrids on the solid support.

The use of heterogenous capture formats for the detection of nucleotide polymorphisms, such as the one described in U.S. Pat. Nos. 5,651,630 and 5,273,882, are also preferred. Heterogenous capture formats employ a capture reagent to separate amplified nucleotide sequences of interest from other materials employed in the amplification reaction. A capture reagent is preferably a solid support material that is coated with one or more specific binding-members, which are specific for the same or a different binding member. The binding member preferably comprises an oligonucleotide that specifically binds with a nucleic acid having a nucleotide sequence of interest. The “solid support material” is any suitable insoluble material, or soluble material that is made insoluble by a subsequent reaction. The solid support material is preferably selected from the group consisting of latex, plastic, derivatized plastic, magnetic metal, non-magnetic metal, glass and silicon. The solid support can have any suitable form or topology and can be a surface of a test tube, microtiter well, sheet, bead, microparticle, chip, or other item. An exemplary capture reagent includes an array that generally comprises oligonucleotides or polynucleotides immobilized to a solid support material in a spatially defined manner. Such an array optionally can be fabricated with a reagent jetting system in accordance with the disclosure of U.S. Pat. No. 4,877,745 to Verlee.

Many heterogeneous detection schemes for differentiating the various signals produced by the various amplification products on the solid support are available. For example, different specific binding members can be employed to bind different amplification products to separate solid supports. Alternatively, all amplification products can be bound to a single solid support but different specific binding members can be employed to selectively bind distinct conjugates to the amplification products such that a different signal is associated with each of the various amplification products.

The haplotype identified can be any haplotype of clinical, research, forensic or other interest. For example, the present invention can be used to determine the combination of TPMT haplotypes of a human whom is suspected of having (or may have) a *3B/*3C TPMT combination of haplotypes. Advantageously, this *3B/*3C combination of TPMT haplotypes, which is clinically relevant, can be distinguished from the *1/*3A combination of TPMT haplotypes, which is essentially innocuous.

The present invention also provides a kit useful, inter alia, in the practice of the present inventive method. The kit comprises a first and second pair of oligonucleotides. The paired oligonucleotides allow amplification of two distinct nucleotide sequences of interest that are known or suspected or have a substantial probability of including a sequence variant of interest. In an embodiment of the inventive kit intended for medical or clinical use, the paired oligonucleotides preferably allow the amplification of known or suspected sequence variants of medical or clinical relevance. Similarly, other embodiments of the present inventive kit can be to amplify the sequence variants of relevance to the particular use for which they are designed.

Each pair of oligonucleotides is able to hybridize to a nucleic acid of the organism at a position at, or near, the site of a putative nucleotide sequence polymorphism or variant under suitably stringent, and preferably highly stringent, conditions. Each oligonucleotide can bind to opposite strands of a double-stranded nucleic acid (e.g., as in a PCR reaction), or can bind to the same strand of a nucleic acid (e.g., as in a LCR reaction). The regions of complementarity of the oligonucleotides to the isogenic nucleotide sequence of interest with respect to a given pair of oligonucleotides preferably do not overlap, but preferably are complementary to sequences on a single chromosome, or to an mRNA and its complement. The sequence of the oligonucleotides preferably does not contain a sequence that is complementary only to the sequence of a specific variant at a polymorphic site, except when LCR reactions, variations thereof, and similar amplification reactions are employed in the present inventive method (wherein it is important for the oligonucleotide to have a sequence that is complementary to the specific allelic sequence at a polymorphism).

The kit preferably also comprises two or more probes that can be used to detect the presence or absence of a nucleotide polymorphism in a test sample or amplification reaction. Suitable probes include those described above and others.

The kit optionally also comprises additional pairs of oligonucleotides and/or additional probes. For example, the kit can comprise a third pair of oligonucleotides that are complementary to a nucleotide sequence flanking a third polymorphic site in an isogenic region of the organism's genome. The third and any additional pairs of oligonucleotides, in embodiments comprising additional pairs of oligonucleotides, can be complementary to the same nucleic acid as the first two pairs or can be complementary to another DNA in the organism's genome (such as would be useful for haplotyping one allele or pair of alleles, and genotyping another allele). Additionally or alternatively, the kit optionally can comprise three or more probes. The third probe (and additional probes beyond a third probe) can either be complementary to the amplification products obtained from a third pair of oligonucleotides or to an additional site in the nucleic acid amplified by the first or second pair of oligonucleotides.

The kit optionally also comprises one or more enzymes useful in the amplification or detection of nucleic acids and/or nucleotide sequences. Suitable enzymes include DNA polymerases, RNA polymerases, ligases, and phage replicases. Additional suitable enzymes include kinases, phosphatases, endonucleases, exonucleases, RNAses specific for particular forms of nucleic acids (including, but not limited to, RNAse H), and ribozymes. Other suitable enzymes can also be included in the kit.

The kit optionally can also comprise other amplification reaction reagents (defined above) as well as detection reaction reagents, such as light or fluorescence generating substrates for enzymes linked to probes. Similarly, the kit optionally can comprise instructions or directions for using the kit in the detection of nucleotide sequence polymorphisms or haplotypes or both.

The kit is preferably provided in a microbiologically stable form. Microbiological stability can be achieved by any suitable means, such as by (i) freezing, refrigeration, or lyophilization of kit components, (ii) by heat-, chemical-, or filtration-mediated sterilization or partial sterilization, and/or (iii) by the addition of antimicrobial agents such as azide, detergents, and other suitable reagents to other kit components. Moreover, the kit is preferably manufactured to meet at least the minimum standards for medical diagnostics set forth by the U.S. Food and Drug Administration, which standards (including but not limited to those standards set forth in the Code of Federal Regulations) as they exist as of the filing date of the present patent specification are specifically incorporated by reference.

The kit can also be optionally provided in a suitable housing that is preferably useful for robotic handling by a clinically-useful sample analyzer. For example, the kit can optionally comprise multiple liquids, each of which are stored in distinct compartments within the housing. In turn, each compartment can be sealed by a device that can be removed, or easily penetrated, by a mechanical device. Each seal isolating the compartments containing liquids of the kit covers an orifice that preferably lies substantially in a single plane or in substantially parallel planes. The alignment of the orifices assists in the efficient aspiration, aliquotting, and/or transfer of kit reagents. The housing can also comprise reaction vessels suitable for aliquotting of liquids, samples and reaction products.

The kit can be incorporated into a present inventive apparatus. The present inventive apparatus comprises the kit and a robotic or automatic sample analyzer. The apparatus can perform one or more steps of the present inventive method, described above. The analyzer is preferably of a suitable design so as to decrease the likelihood of cross-contamination of samples. Suitable features of design include the use of aspiration barriers, disposable surfaces, and other means.

The kit can also be configured to be used in any other embodiments of the present inventive method described above.

The following example further illustrates the present invention but should not be construed as limiting its scope in any way.

### EXAMPLE

This example illustrates the use of the present invention to distinguish the human *1/*3A combination of TPMT haplotypes from the *3B/*3C combination of TPMT haplotypes.

In this example, the organism is a human known to have two nucleotide sequence variants in the TPMT locus that can interfere with thiopurine metabolism. From this initial information, an ordinarily skilled medical geneticist can infer that the individual is either of the *1/*3A haplotype combination or the *3B/*3C haplotype combination. The difference is important to the appropriate clinical treatment of the individual.

Three micrograms of whole DNA are extracted from the human and placed in a reaction vessel. Oligonucleotides that are complementary to nucleotide sequences flanking these TPMT polymorphisms are added to the extracted whole DNA such that the final concentration of each added oligonucleotide is about 100 nanomolar. The resultant solution comprises about 10⁶ copies of the human genome. The sample is serially diluted in 96-well microtiter plates adapted for thermocycling such that, after serial dilution across the wells of the plates, one plate with the following contents: 10 wells comprise by calculation 1.7 copies of the genome per well, 10 wells comprise by calculation 0.8 copies of the genome per well, 10 wells comprise by calculation 0.6 copies of the genome, 10 wells comprise 0.5 copies of the genome, and 10 wells comprise 0.25 copies of the genome. The serial dilution is performed in a reaction mix comprising heat-activatable thermostable DNA polymerase and all the other components for duplex PCR other than the target DNA and oligonucleotide amplimers.

The reactions are submitted to 20 cycles of PCR under suitable time and temperature parameters, and with a relatively low level of amplification reactants suitable for the first stage of the amplification technique known as “booster PCR.” After the initial 20 cycles of PCR, additional oligonucleotides and amplification reaction components are added such that the concentration of each oligonucleotide amplimer is about 100 nanomolar. PCR is then carried out for an additional 50 cycles, wherein the elongation times can be slightly shorter. The PCR reactions are then cooled to about 8° C., which substantially stops the DNA amplification reaction.

A portion of each amplification reaction is then mixed individually (i.e., separately) with a molecular beacon probe specific for each of the TPMT nucleotide sequence polymorphisms constituting the *3A genotype. Alternatively, amplification primers & molecular beacon probes can be added at one time and the presence or absence of specific alleles can be carried out simultaneously with the amplification step. As is well known in the art, the *3A haplotype consists of a single chromosome comprising the nucleotide sequence variants which if present alone constitute the *3B haplotype and the *3C haplotype.

Molecular beacon probes, which are known in the art, comprise a fluorescence emitter and fluorescence quencher. When the probe is not hybridized to a target sequence, the emitted fluorescence is low. In contrast, when the probe hybridizes with a target sequence the emission of fluorescence is greatly enhanced thereby indicating the presence of the target sequence. Because fluorescent emissions have distinctive colors, two or more molecular beacons can be added to a single sample and the ordinarily skilled artisan can readily determine the extent of binding by each beacon. Accordingly, a control beacon that is specific for a non-polymorphic region of the TPMT gene and has a different color than the other molecular beacons is also added to each mixture of amplification reaction and polymorphism-specific molecular beacon probe. The control beacon allows the ordinarily skilled artisan to detect whether nucleic acid amplification occurred in any particular test location has occurred. However, the use of a control probe or molecular beacon is optional.

Each test location is then scored for the presence of amplification products, and the presence or absence of each polymorphism. By observation of the scored wells, the skilled artisan can readily infer which row of ten wells comprises individual wells in which only 0 or 1 copy of the isogenic sequence of interest was amplified.

If the *3B and *3C polymorphisms substantially always appear together, then the human is *1/*3A and able to safely metabolize azathiaprine. If the *3B and *3C polymorphisms both appear, but never or rarely in the same test location, then the human is *3B/*3C and high quantities of azathiaprine would be expected to have an adverse clinical impact, whereas low quantities (i.e., quantities normally considered sub-therapeutic in *1/*3A patients) may be usefully administered to the patient. Advantageously, these haplotypes can be distinguished from each other in a single day and without multiple patient-physician interactions.

In this prophetic example, the 10 wells calculated to contain an average of 0.5 genome equivalents per well were scored. Five wells contained no detectable amplification products; 3 wells contained the nucleotide sequence characteristic of the *3B haplotype, but not the *3C (and thus also not the *3A) haplotype; and 2 wells contained the nucleotide sequence characteristic of the *3C haplotype, but no the *3B (and thus not the *3A) haplotype. Accordingly, the human has the *3B/*3C combination of TPMT haplotypes, and probably (with discretion left to the treating physician) should not be administered high concentrations of azathiaprine. The remaining wells were not scored because the 10 wells calculated to contain an average of 0.5 genome equivalents per well yielded satisfactory data.

All of the references cited herein, including patents, patent applications, and references, are hereby incorporated in their entireties by reference.

While this invention has been described with an emphasis upon preferred embodiments, it will be obvious to those of ordinary skill in the art that variations of the preferred embodiments can be used and that it is intended that the invention can be practiced otherwise than as specifically described herein. Accordingly, this invention includes all modifications encompassed within the spirit and scope of the invention as defined by the following claims.

What is claimed is:

1. A method of identifying the haplotype of an organism, the method comprising:
(a) providing a sample comprising nucleic acids from the organism, wherein the nucleic acids comprise at least two copies of an isogenic nucleotide sequence of interest,
(b) aliquotting the nucleic acids into test locations such that at least one test location is expected to contain one, and only one, isogenic nucleotide sequence of interest,
(c) amplifying the isogenic nucleotide sequence of interest in a predetermined number of test locations to create amplification products,
(i) wherein amplifying the isogenic nucleotide sequence of interest employs two pairs of oligonucleotide primers
(ii) such that at least one test location is expected to contain amplification products having a unique nucleotide sequence corresponding to the nucleotide sequence of one, and only one, of the isogenic nucleotide sequences of interest in the organism's genome, and
(d) detecting the presence or absence of specific forms of a first nucleotide polymorphism and a second nucleotide polymorphism in the isogenic region of interest at two non-contiguous positions in the nucleotide sequence of interest by detecting the presence or absence of specific forms of the first nucleotide polymorphism and second nucleotide polymorphism in the amplification products in each of the predetermined number of test locations comprising amplified nucleic acids,
such that the haplotype of the organism is identified.

2. The method of claim 1, wherein on average less than about 1 copy of the isogenic region of interest is aliquotted into each test location.

3. The method of claim 2, wherein on average less than about 0.67 copies of the isogenic region of interest is aliquotted into each test location.

4. The method of claim 2, wherein on average from about 0.4 copies to about 0.6 copies of the isogenic region of interest is aliquotted into each test location.

5. The method of claim 1, wherein the step of amplifying the isogenic region of interest employs a duplexed or multiplexed method selected from the group consisting of Qu replicase mediated amplification, ligase chain reaction, NASBA, and transcription mediated amplification.

6. The method of claim 1, wherein the step of amplifying the isogenic region of interest employs multiplexed polymerase chain reaction.

7. The method of claim 1, wherein
at least about 1 kilobase of nucleotide sequence in the isogenic region of interest separates the nucleotide sequences in the isogenic region of interest that are complementarity to an oligonucleotide primer of (i) the first oligonucleotide primer pair and (ii) an oligonucleotide primer of the second primer pair.

8. The method of claim 7, wherein
at least about 5 kilobases of nucleotide sequence in the isogenic region of interest separates the nucleotide sequences in the isogenic region of interest that are complementarity to an oligonucleotide primer of (i) the first oligonucleotide primer pair and (ii) an oligonucleotide primer of the second primer pair.

9. The method of claim 1, wherein the step of detecting the absence or presence of a particular polymorphism employs a probe.

10. The method of claim 9, wherein said probe is a molecular beacon probe.

11. The method of claim 1, wherein the sample comprising nucleic acids from the organism comprises genomic DNA.

12. The method of claim 1, wherein the sample comprising nucleic acids from the organism comprises cDNA.

13. The method of claim 1, wherein the sample comprising nucleic acids from the organism comprises isogenic polymerase chain reaction products.

14. The method of claim 1, wherein a test location is a well of a multi-well test plate.

15. The method of claim 1, wherein a test location is an isolated position in an array.

16. The method of claim 15, wherein the array is formed by a reagent jetting system.

17. The method of claim 1, wherein the organism is human and the haplotype identified is a TPMT haplotype.

18. The method of claim 17, wherein the method distinguishes between the *1/*3A and *3B/*3C combinations of haplotypes.

19. A kit, useful for identifying the haplotype of an organism having a diploid genome, comprising:
(a) a first pair of oligonucleotides,
(b) a second pair of oligonucleotides, wherein
(i) the first pair of oligonucleotides are complementary to a nucleotide sequence flanking a first polymorphism in an isogenic region of the organism's genome
(ii) the second pair of oligonucleotides are complementary to a nucleotide sequence flanking a second polymorphic site in an isogenic region of the organism's genome
(iii) no oligonucleotide of the first pair or second pair of oligonucleotides is complementary to a nucleotide sequence in the isogenic nucleotide sequence of interest that is complementary to another oligonucleotide of the first pair or second pair of oligonucleotides
(c) a first probe specific for the first polymorphism within a first isogenic nucleotide sequence of interest, and
(d) a second probe specific for the second polymorphism within a second isogenic nucleotide sequence of interest.

20. The kit of claim 19 further comprising an enzyme selected from the group consisting of DNA polymerases, RNA polymerases, ligases, and phage replicases.

21. The kit of claim 19 further comprising a third pair of oligonucleotides, wherein the third pair of oligonucleotides are complementary to a nucleotide sequence flanking a third polymorphic site in an isogenic region of the organism's genome.

22. A kit, useful for identifying the haplotype of an organism having a diploid genome, comprising:
(a) a first pair of oligonucleotides,
(b) a second pair of oligonucleotide, wherein
(i) the first pair of oligonucleotides are complementary to a nucleotide sequence flanking a first polymorphism in an isogenic region of the organism's genome
(ii) the second pair of oligonucleotides are complementary to a nucleotide sequence flanking a second polymorphic site in an isogenic region of the organism's genome
(iii) no oligonucleotide of the first pair or second pair of oligonucleotides is complementary to a nucleotide sequence in the isogenic nucleotide sequence of interest that is complementary to another oligonucleotide of the first pair or second pair of oligonucleotides
(c) a means of detecting one or more specific forms of a first polymorphism within a first isogenic nucleotide sequence of interest, and
(d) a means of detecting one or more specific forms of a second polymorphism within a second isogenic nucleotide sequence of interest.
